(19) United States
(12) Patent Application Publication
(10) Pub. No.: US 2018/0160998 A1
(43) Pub. Date: Jun. 14, 2018

(54) ULTRASOUND SYSTEM AND METHOD FOR VISUALIZING THE FLOW OF A LIQUID WITHIN AN OBJECT

(71) Applicant: GENERAL ELECTRIC COMPANY, SCHENECTADY, NY (US)

(72) Inventors: DANIEL LAGLER, ZIPF (AT); EWALD LAGLER, ZIPF (AT)

(73) Assignee: GENERAL ELECTRIC COMPANY, SCHENECTADY, NY (US)

(21) Appl. No.: 15/376,976

(22) Filed: Dec. 13, 2016

(51) Int. Cl. A61B 8/06; A61B 8/00; A61B 8/08

(52) U.S. Cl. CPC A61B 8/06; A61B 8/463; A61B 8/5207; A61B 8/4444; A61B 8/5269

(57) ABSTRACT

An ultrasound system for visualizing the flow of a liquid within an object is provided. The ultrasound system includes an ultrasound probe, a generating unit, and a display processing unit. The ultrasound probe is operative to obtain ultrasound data from the object. The generating unit is operatively connected with the ultrasound probe and configured to render one or more images based at least in part on the ultrasound data. Each image includes a flow artifact that depicts the flow of the liquid. The display processing unit is operatively connected with the generating unit and configured to display the one or more images rendered by the generating unit. The generating unit modulates a property of the flow artifact based at least in part on a flow characteristic of the liquid.

### BACKGROUND

### Technical Field

Embodiments of the invention relate generally to ultrasound technologies, and more specifically, to an ultrasound system and method for visualizing the flow of a liquid within an object.

### Discussion of Art

Generally, ultrasound medical imaging is used to survey internal structures for diagnostic purposes. Ultrasound imaging has comparatively short acquisition times per image, on the order of one tenth of a second as compared to several seconds for Magnetic Resonance Imaging (“MRI”), can acquire many images with minimal patient risk, and offers a unique role for an ultrasound technician as part of the imaging system control loop. It therefore is particularly used for imaging moving internal structures, e.g., for imaging blood flow within a patient.

While many ultrasound systems are capable of producing images of blood flowing within a patient, such ultrasound systems typically depict blood flow in a limited manner. For example, many such ultrasound systems only depict a single flow characteristic of the blood, e.g., the velocity of the blood flow in an axial direction, in a vessel and/or internal organ. In order to quickly and accurately diagnose many medical conditions, however, medical physicians often require information regarding additional flow characteristics, e.g., the direction and magnitude of velocity, the direction and magnitude of acceleration, turbulence, and/or strain.

What is needed, therefore, is an improved ultrasound system and method for visualizing the flow of a liquid within an object.

### BRIEF DESCRIPTION

In an embodiment, an ultrasound system for visualizing the flow of a liquid within an object is provided. The ultrasound system includes an ultrasound probe, a generating unit, and a display processing unit. The ultrasound probe is operative to obtain ultrasound data from the object. The generating unit is operatively connected with the ultrasound probe and configured to render one or more images based at least in part on the ultrasound data. Each image includes a flow artifact that depicts the flow of the liquid. The display processing unit is operatively connected with the generating unit and configured to display the one or more images rendered by the generating unit. The generating unit modulates a property of the flow artifact based at least in part on a flow characteristic of the liquid.

In another embodiment, a method for visualizing the flow of a liquid within an object is provided. The method includes: obtaining ultrasound data from the object via an ultrasound probe; rendering one or more images based at least in part on the ultrasound data via a generating unit operatively connected to the ultrasound probe, each image including a flow artifact that depicts the flow of the liquid; displaying the one or more rendered images on a display processing unit operatively connected with the generating unit; and modulating a property of the flow artifact based at least in part on a flow characteristic of the liquid.

In yet another embodiment, a non-transitory computer readable medium is provided. The non-transitory computer readable medium stores instructions configured to adapt a generating unit, of an ultrasound system for visualizing a liquid within an object, that includes at least one processor and a memory device to: obtain ultrasound data from the object via an ultrasound probe operatively connected with the generating unit; render one or more images based at least in part on the ultrasound data, each image including a flow artifact that depicts the flow of the liquid; and modulate a property of the flow artifact based at least in part on a flow characteristic of the liquid; and display the one or more rendered images on a display processing unit operatively connected with the generating unit. The flow artifact is at least one of a pathline, a streamline, a ribbon, and a contour map, and the flow characteristic of the liquid is at least one of velocity direction, velocity magnitude, acceleration direction, acceleration magnitude, power, turbulence, and strain.

### DRAWINGS

The present invention will be better understood from reading the following description of non-limiting embodiments, with reference to the attached drawings, wherein below:

FIG. 1 is a perspective view of an ultrasound system for visualizing the flow of a liquid within an object in accordance with an embodiment of the invention;

FIG. 2 is a view of an image generated by the ultrasound system of FIG. 1 in accordance with an embodiment of the invention;

FIG. 3 is another view of an image generated by the ultrasound system of FIG. 1 in accordance with an embodiment of the invention;

FIG. 4 is another view of an image generated by the ultrasound system of FIG. 1 in accordance with an embodiment of the invention; and

FIG. 5 is another view of an image generated by the ultrasound system of FIG. 1 in accordance with an embodiment of the invention.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts, without duplicative description.

As used herein, the terms “substantially,” “generally,” and “about” indicate conditions within reasonably achievable manufacturing and assembly tolerances, relative to ideal desired conditions suitable for achieving the functional purpose of a component or assembly. As used herein, “electrically coupled”, “electrically connected”, and “electrical communication” mean that the referenced elements are directly or indirectly connected such that an electrical current may flow from one to the other. The connection may include a direct conductive connection, i.e., without an intervening capacitive, inductive or active element, an inductive connection, a capacitive connection, and/or any other suitable electrical connection. Intervening components may be present. The term “real-time,” as used herein, means a level of processing responsiveness that a user senses as sufficiently immediate or that enables the processor to keep up with an external process.

Further, while the embodiments disclosed herein are described with respect to ultrasound systems and images, it is to be understood that embodiments of the present invention may be applicable to other types of imaging capable of visualizing the flow of a liquid within an object, e.g., Magnetic Resonance Imaging (“MRI”), x-ray imaging, etc. Further still, as will be appreciated, embodiments of the present invention related imaging systems may be used to analyze tissue generally and are not limited to human tissue.

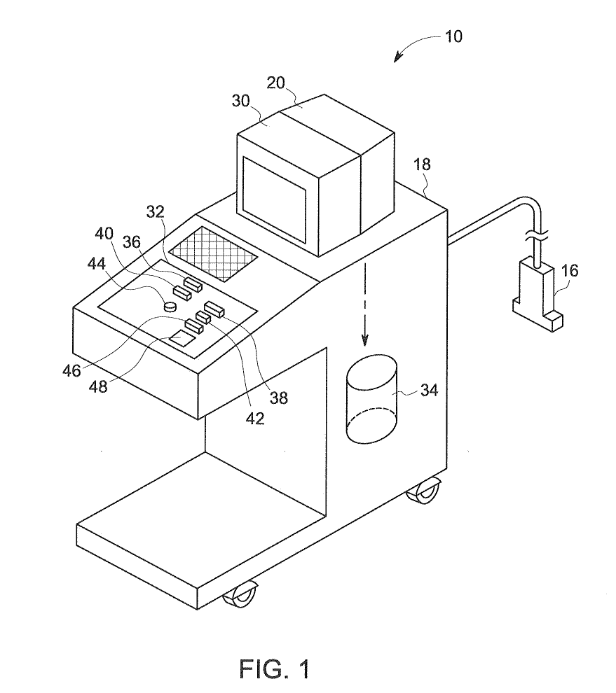

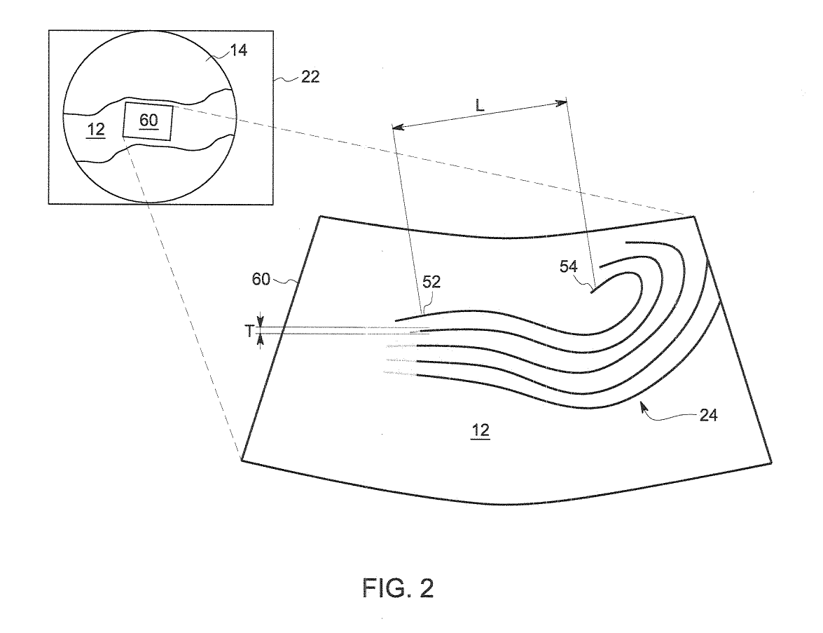

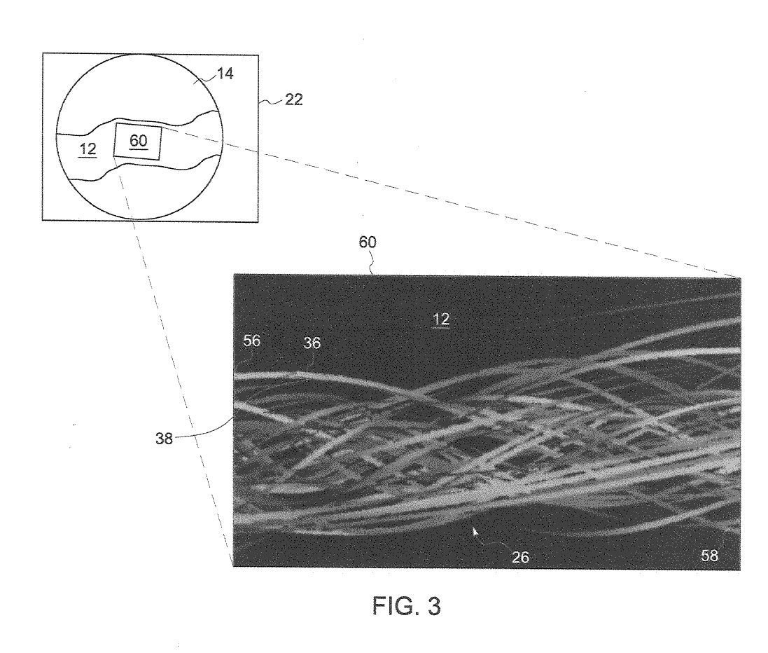

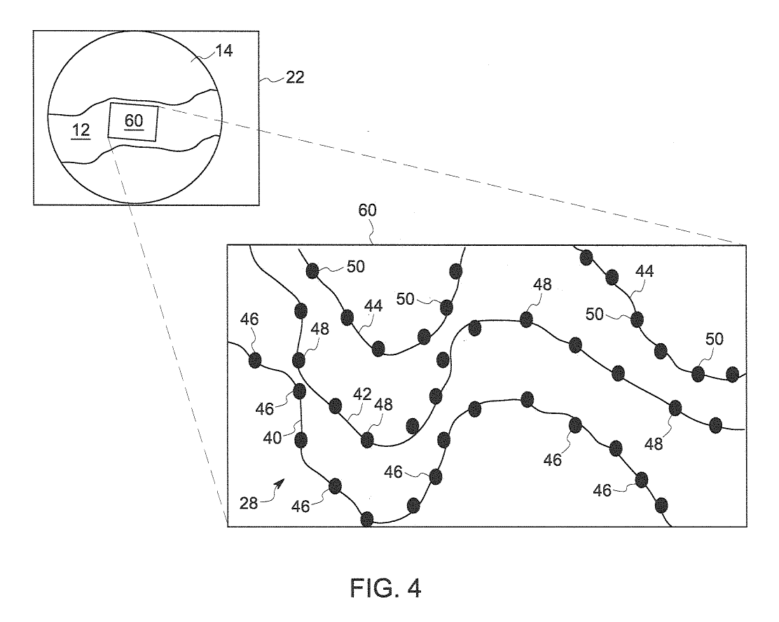

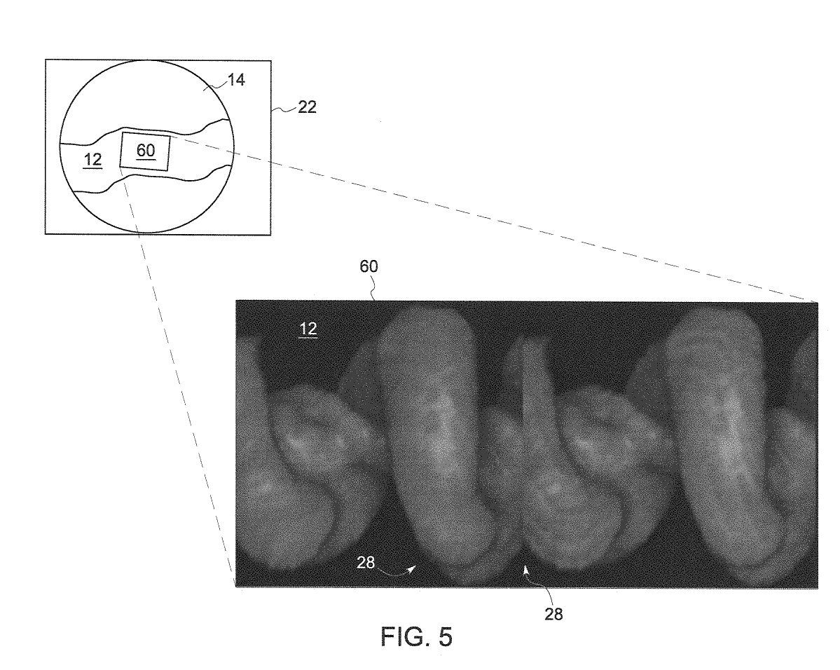

Referring now to FIG. 1, the major components of a system 10 for visualizing the flow of a liquid 12 (FIGS. 2-4), e.g., blood, within an object 14 (FIGS. 2-4), e.g., a patient, are shown. The system 10 includes an ultrasound probe 16, generating unit 18, and a display processing unit 20. The ultrasound probe 16 is operative to obtain ultrasound data from the object 14. The generating unit 18 is operatively connected with the ultrasound probe 16 and is configured to render one or more images 22 (FIGS. 2-4) based at least in part on the ultrasound data. As will be appreciated, the one or more images 22 may be still frames and/or registered together to form a moving image, e.g., video. Each of the images 22 includes a flow artifact 24 (FIG. 2), 26 (FIG. 3), and 28 (FIGS. 4 and 5), that depicts the flow of the liquid 12. As used herein, the term “flow artifact” means a rendered graphical objected that conveys information about a flow characteristic, e.g., velocity direction, velocity magnitude, acceleration direction, acceleration magnitude, power, turbulence, and strain, of a flow unit of the liquid 12 within the object. As used herein, the term “flow unit” means an amount of the liquid 12 used to model and/or measure the flow of the liquid 12. The display unit 20 is operatively connected with the generating unit 18 and is configured to display the one or more images 22 rendered by the generating unit 18. As will be explained in greater detail below, the generating unit 18 modulates a property of the flow artifact 24, 26, 28 based at least in part on a flow characteristic of the liquid 12, e.g., velocity, acceleration, power, turbulence, and/or strain.

In embodiments, the ultrasound probe 16 transmits an ultrasonic pulse into the object 14, e.g., the body of a human patient, and receives an ultrasonic echo from within the object 14. For example, the ultrasound probe 16 emits one or more ultrasonic pulses scheduled at a pulse repetition frequency (“PRF”) and recovers ultrasonic echo signals that are returned from the object to a plurality of two-dimensionally distributed sampling points. As will be understood, the ultrasound probe 16 transduces the ultrasonic echo signals into digital data that is sent to the generating unit 18.

As stated above, the generating unit 18 renders/generates the ultrasound images 22 on the basis of the ultrasonic echo. The images 22 are then displayed on a monitor 30 of the display processing unit 20. In embodiments, the generating unit 18 includes an operation panel 32 that accepts instructions from an operator (not shown), and a storage device 34, e.g., a hard drive, that stores the ultrasound images 22 and other data obtained/measured from the images 22. The operation panel 32 includes ultrasound imaging control, e.g., an image list button 36, a record button 38, an image pickup condition recall button 40, a display zoom button 42, a freeze button 44, a position record button 46, and a cursor track ball 48.

Turning now to FIG. 2, as stated above, each of the images 22 includes a flow artifact 24 that depicts the flow of the liquid 12. As shown in FIG. 2, in embodiments, the flow artifact 24 may be one or more lines, which may be pathlines and/or streamlines. As will be understood, a pathline represents the actual path of a flow unit over a duration of time, and may be modeled for a given velocity field function ū(x̅, t) and an initial position x̅₀̅ by the following equation:

x̅ (x̅₀̅, t)=x̅₀̅+∫ₜ_{₀}^{t}ū(x̅(x̅₀̅,τ),τ)⋅dτ

wherein x̅(x̅₀̅, t,) represents the pathline. As will be further understood, a streamline is an imaginary line which is tangential to the velocity vector for each position at a specific point in time, for a given flow unit, and can be expressed as a function of a curve parameter s for a given velocity field function ū(x̅, t), an initial position x̅₀̅, and a point in time t by the following equation:

x̅(x̅₀̅t, s)=x̅₀̅+∫ₛ_{₀}^{s}ū(x̅(x̅₀̅, t, ç), ç)⋅dç

wherein x̅(x̅₀̅, t, s) represents the streamline.

In embodiments, the above pathline and streamline functions can be numerically approximated by different discretization methods, e.g., Euler, Runge-Kutta, etc. For example, a forward Euler method for solving the above pathline equation is as follows:

x̅ₜ̅₊̅₁̅=x̅ₜ̅+u̅ₜ̅⋅Δt

wherein t denotes the time step iteration number and Δt specifies a certain time step size. Additionally, in embodiments, the above streamline function can be solved recursively as follows:

x̅ₛ̅₊̅₁̅=x̅_̅{̅w̅}̅+u̅ₛ̅⋅s

wherein the iteration is done spatially over s using a step size of Δs.

As will be appreciated, a velocity of a flow unit is generally not constant during the time interval of the iteration step in the above solutions, thus, higher order approximations generally lead to better results. Further, in embodiments, the time step size may be constant. Accordingly, in some embodiments, an adaptive approach for choosing the step size may improve the quality of the results. For example, in such embodiments, the step size may be increased for small velocity gradients and vice versa. Additionally, interpolation methods, e.g., polynomial, Hermite, Bézier, B-spline, etc., may be utilized in some embodiments, where it may not be possible to set the step size small enough to achieve a smooth curve, so as to visually provide the expression of a continuous representation. Further, while the pathlines and streamlines 24 are depicted herein as solid lines, it is to be understood that, in other embodiments, the pathlines and/or streamlines 24 may be depicted as dashed lines, dots, arrows, circles, tubes, and/or any other appropriate shape capable of conveying the movement of flow units of the liquid 12 within the object 14.

As illustrated in FIG. 3, in embodiments, the flow artifact 26 may be one or more ribbons. As will be understood, the ribbons 26 provide for the visualization of the twisting of the liquid 12 moving through the object 14, and in embodiments, may be based at least in part on pathlines and/or streamlines 24, e.g., the data from which pathlines and/or streamlines 24 may be constructed from. For example, each ribbon 26 may have a first side 36 and a second side 38, wherein the first side 36 represents the path a first flow unit and the second side 38 represents the path of second flow unit spaced apart from the first flow unit. Thus, the “twists” in the ribbons 26 depict the movements/paths of the first flow unit and the second flow unit with respect to each other.

Referring now to FIGS. 4 and 5, in embodiments, the flow artifact 28 may be a contour/relievo map. As will be appreciated, in embodiments, the contour map be two dimensional (“2D”) or three dimensional (“3D”), as shown in FIGS. 4 and 5, respectively. As shown in FIG. 4, in embodiments where the contour map is 2D, the contour map may include a plurality of lines 40, 42, 44 that connect a plurality of dots 46, 48, 50 representing flow units having at least one shared flow characteristics. As shown in FIG. 5, in embodiments where the contour map is 3D, the contour map may include a plurality of meshes/surfaces each defined by a plurality of vertices representing flow units having at least one shared flow characteristic. Additionally, the color and/or fading of the lines 40, 42, 44 (for 2D contour maps) and of the surfaces (for 3D contour maps) may be varied to reflect additional flow characteristics.

Further, in embodiments, the generating unit 18 may implement a smoothing filter in order to smooth the appearance of the rendered artifacts 24, 26, 28.

Returning back to FIG. 2, as stated above, in embodiments, the generating unit 18 modulates a property of the flow artifact 24 based at least in part on a flow characteristic. As used herein, modulation of a property of a flow artifact 24, 26, 28 means altering and/or adjusting a visual appearance and/or rendering property, e.g., color, length, thickness, etc, of the flow artifact 24, 26, 28. For example, in embodiments, the modulated property may be a thickness T of the pathlines and/or streamlines 24. For example, in embodiments, pathlines and/or stream lines 24 having higher velocity magnitudes and/or acceleration magnitudes may be thicker than pathlines and/or streamlines 24 having lower velocity magnitudes and/or acceleration magnitudes, or vice versa.

In embodiments, the modulated property may be a length of the pathlines and/or streamlines 24. For example, in embodiments, pathlines and/or stream lines 24 having higher velocity magnitudes and/or acceleration magnitudes may be longer than pathlines and/or streamlines 24 having lower velocity magnitudes and/or acceleration magnitudes, or vice versa. As will be appreciated, in embodiments, the length of the ribbons 26 may be modulated in a similar manner.

In embodiments, the modulated property may be alpha fading of a tail 52 of a pathline and/or streamline 24, as compared to a head 54 of the pathline and/or streamline 24. As used herein, alpha fading refers to the alteration of the transparency/opacity and/or coloration of a part, e.g., the tail end 54, of a rendered object, e.g., the lines 24, within the images 22. For example, in embodiments, pathlines and/or stream lines 24 having lower velocity magnitudes and/or acceleration magnitudes have higher alpha fading on their tails 52 than pathlines 24 and/or streamlines 24 having higher velocity magnitudes and/or acceleration magnitudes, or vice versa. As will be appreciated, in embodiments, the ribbons 26 may have tail ends 56 alpha faded so that they appear different from heads 58 of the ribbons 26 in a manner similar to the lines 24 as discussed above.

In embodiments, the flow artifact 24, 26, and 28 may be based at least in part on a time to live duration t_{d}, which as used herein, refers to an amount of time depicted within the images 22. For example, in embodiments, the tail 52, 56 and head 54, 58 of a line 24 or a ribbons 26 may represent a flow unit at an initial time t₀ and at the expiration of the time to live duration t_{d}, respectively. In other words, in embodiments, a flow artifact 24, 26, and 28 reflects the flow characteristics of one or more flow units of the liquid 12 within the object 14 from t₀to (t₀+t_{d}). As will be appreciated, the time to live duration t_{d} may be based at least in part on a velocity of the liquid 12. For example, in embodiments, the time to live duration t_{d} may be longer for low velocity magnitudes, i.e., slow flow, then it is for high velocity magnitudes, i.e., fast flow.

As further shown in FIG. 2, the flow artifact 24, 26, 28 may be based at least in part on a selection window 60. As will be appreciated, the selection window 60 defines a region in the one or more images 22 within which the flow artifact 24, 26, 28 is rendered. For example, an operator may input the selection window 60 into the generating unit 18 via a user interface provided by the display processing unit 20. The generating unit 18 then renders the flow artifact 24, 26, 28 such that the flow artifact 24, 26, 28 represents/depicts the flow of the liquid 12 through the region of the object 14 shown in the images as being within the selection window 60. In certain embodiments, the selection window 60 may define the location of the tails 52, 56 of the lines 24 and/or the ribbons 26 at t₀ and the generating unit 18 may then render as much of the lines 24 and/or ribbons 26 as necessary to depict the flow of the corresponding flow units throughout the time to live duration t_{d}. In other words, the pathlines and/or streamlines 24 may also be timelines. In aspects, the selection window 60 may allow an operator and/or physician to focus on a small region within the object/patient 14 by limiting the flow units of the liquid/blood modeled by the flow artifacts 24, 26, 28. For example, the selection window 60 may be between about 2 cm-by-2 cm to 8 cm-by-8 cm. The selection window 60 may be selected/defined prior to, and/or during, the obtaining of ultrasound data via the ultrasound probe 16, i.e., the ultrasound system 10 provides for real-time rendering of the flow artifacts 24, 26, 28. As will be appreciated, however, in other embodiments, the selection window 60 may be selected/defined after the ultrasound data has been obtained by the ultrasound probe 16, i.e., the ultrasound system 10 provides for the ability to render the flow artifacts 24, 26, 28 within the images 22 utilizing pre-recorded/captured ultrasound data. Further, while the selection window 60 is depicted in the accompanying figures as a rectangle, in embodiments, the selection window 60 may have a triangular, circular, and/or other shapes, to include operator defined shapes, that define a region within the object 14 through which the liquid 12 flows.

Finally, it is also to be understood that the system 10 may include the necessary electronics, software, memory, storage, databases, firmware, logic/state machines, microprocessors, communication links, displays or other visual or audio user interfaces, printing devices, and any other input/output interfaces to perform the functions described herein and/or to achieve the results described herein. For example, as previously mentioned, the system may include at least one processor and system memory/data storage structures, which may include random access memory (RAM) and read-only memory (ROM). The at least one processor of the system 10 may include one or more conventional microprocessors and one or more supplementary co-processors such as math co-processors or the like. The data storage structures discussed herein may include an appropriate combination of magnetic, optical and/or semiconductor memory, and may include, for example, RAM, ROM, flash drive, an optical disc such as a compact disc and/or a hard disk or drive.

Additionally, a software application that adapts the controller to perform the methods disclosed herein may be read into a main memory of the at least one processor from a computer-readable medium. The term “computer-readable medium”, as used herein, refers to any medium that provides or participates in providing instructions to the at least one processor of the system 10 (or any other processor of a device described herein) for execution. Such a medium may take many forms, including but not limited to, non-volatile media and volatile media. Non-volatile media include, for example, optical, magnetic, or opto-magnetic disks, such as memory. Volatile media include dynamic random access memory (DRAM), which typically constitutes the main memory. Common forms of computer-readable media include, for example, a floppy disk, a flexible disk, hard disk, magnetic tape, any other magnetic medium, a CD-ROM, DVD, any other optical medium, a RAM, a PROM, an EPROM or EEPROM (electronically erasable programmable read-only memory), a FLASH-EEPROM, any other memory chip or cartridge, or any other medium from which a computer can read.

While in embodiments, the execution of sequences of instructions in the software application causes at least one processor to perform the methods/processes described herein, hard-wired circuitry may be used in place of, or in combination with, software instructions for implementation of the methods/processes of the present invention. Therefore, embodiments of the present invention are not limited to any specific combination of hardware and/or software.

It is further to be understood that the above description is intended to be illustrative, and not restrictive. For example, the above-described embodiments (and/or aspects thereof) may be used in combination with each other. Additionally, many modifications may be made to adapt a particular situation or material to the teachings of the invention without departing from its scope.

For example, in an embodiment, an ultrasound system for visualizing the flow of a liquid within an object is provided. The ultrasound system includes an ultrasound probe, a generating unit, and a display processing unit. The ultrasound probe is operative to obtain ultrasound data from the object. The generating unit is operatively connected with the ultrasound probe and configured to render one or more images based at least in part on the ultrasound data. Each image includes a flow artifact that depicts the flow of the liquid. The display processing unit is operatively connected with the generating unit and configured to display the one or more images rendered by the generating unit. The generating unit modulates a property of the flow artifact based at least in part on a flow characteristic of the liquid. In certain embodiments, the flow artifact is at least one of a pathline, a streamline, a ribbon, and a contour map. In certain embodiments, the flow characteristic of the liquid is at least one of velocity direction, velocity magnitude, acceleration direction, acceleration magnitude, power, turbulence, and strain. In certain embodiments, the flow artifact is at least one of a pathline and a streamline, and the modulated property is a thickness of the flow artifact. In certain embodiments, the flow artifact is at least one of a pathline, a streamline, and a ribbon, and the modulated property is a length of the flow artifact. In certain embodiments, the flow artifact is at least one of a pathline, a streamline, and a ribbon, and the modulated property is an alpha fading of a tail end of the flow artifact. In certain embodiments, the flow artifact is at least one of a pathline, a streamline, and a ribbon, and the flow artifact is based at least in part on a time to live duration. In certain embodiments, the time to live duration is based at least in part on a velocity of the liquid. In certain embodiments, the flow artifact is based at least in part on a selection window. In certain embodiments, the object is a patient and the liquid is the blood of the patient.

Other embodiments provide for a method for visualizing the flow of a liquid within an object. The method includes: obtaining ultrasound data from the object via an ultrasound probe; rendering one or more images based at least in part on the ultrasound data via a generating unit operatively connected to the ultrasound probe, each image including a flow artifact that depicts the flow of the liquid; displaying the one or more rendered images on a display processing unit operatively connected with the generating unit; and modulating a property of the flow artifact based at least in part on a flow characteristic of the liquid. In certain embodiments, the flow artifact is at least one of a pathline, a streamline, a ribbon, and a contour map. In certain embodiments, the flow characteristic of the liquid is at least one of velocity direction, velocity magnitude, acceleration direction, acceleration magnitude, power, turbulence, and strain. In certain embodiments, the flow artifact is at least one of a pathline and a streamline, and the modulated property is a thickness of the flow artifact. In certain embodiments, the flow artifact is at least one of a pathline, a streamline, and a ribbon, and the modulated property is a length of the flow artifact. In certain embodiments, the flow artifact is at least one of a pathline, a streamline, and a ribbon, and the modulated property is an alpha fading of a tail end of the flow artifact. In certain embodiments, the method further includes determining a time to live duration via the generating unit. In such embodiments, the flow artifact is at least one of a pathline, a streamline, and a ribbon, and the flow artifact is based at least in part on the time to live duration. In certain embodiments, determining a time to live duration via the generating unit is based at least in part a velocity of the liquid. In certain embodiments, the method further includes selecting a selection window. In such embodiments, the flow artifact is based at least in part on the selection window.

Yet still other embodiments provide for a non-transitory computer readable medium. The non-transitory computer readable medium stores instructions configured to adapt a generating unit, of an ultrasound system for visualizing a liquid within an object, that includes at least one processor and a memory device to: obtain ultrasound data from the object via an ultrasound probe operatively connected with the generating unit; render one or more images based at least in part on the ultrasound data, each image including a flow artifact that depicts the flow of the liquid; and modulate a property of the flow artifact based at least in part on a flow characteristic of the liquid; and display the one or more rendered images on a display processing unit operatively connected with the generating unit. The flow artifact is at least one of a pathline, a streamline, a ribbon, and a contour map, and the flow characteristic of the liquid is at least one of velocity direction, velocity magnitude, acceleration direction, acceleration magnitude, power, turbulence, and strain.

Accordingly, as will be appreciated, by modulating a property of one or more flow artifacts based at least in part on a flow characteristic of the liquid, some embodiments of the invention provide for the ability to convey more information, e.g., velocity direction, velocity magnitude, acceleration direction, acceleration magnitude, strain, turbulence, etc., within rendered ultrasound images than traditional systems. Accordingly, some embodiments of the invention provide for improved ultrasound diagnostics. Further, some embodiments may provide for a more atheistically pleasing and/or interesting presentation of the flow of blood within a patient.

Additionally, while the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, terms such as “first,” “second,” “third,” “upper,” “lower,” “bottom,” “top,” etc. are used merely as labels, and are not intended to impose numerical or positional requirements on their objects. Further, the limitations of the following claims are not written in means-plus-function format are not intended to be interpreted as such, unless and until such claim limitations expressly use the phrase “means for” followed by a statement of function void of further structure.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

Since certain changes may be made in the above-described invention, without departing from the spirit and scope of the invention herein involved, it is intended that all of the subject matter of the above description shown in the accompanying drawings shall be interpreted merely as examples illustrating the inventive concept herein and shall not be construed as limiting the invention.

What is claimed is:

1. An ultrasound system for visualizing the flow of a liquid within an object comprising:
an ultrasound probe operative to obtain ultrasound data from the object;
a generating unit operatively connected with the ultrasound probe and configured to render one or more images based at least in part on the ultrasound data, each image including a flow artifact that depicts the flow of the liquid; and
a display processing unit operatively connected with the generating unit and configured to display the one or more images rendered by the generating unit; and
wherein the generating unit modulates a property of the flow artifact based at least in part on a flow characteristic of the liquid.

2. The ultrasound system of claim 1, wherein the flow artifact is at least one of a pathline, a streamline, a ribbon, and a contour map.

3. The ultrasound system of claim 1, wherein the flow characteristic of the liquid is at least one of velocity direction, velocity magnitude, acceleration direction, acceleration magnitude, power, turbulence, and strain.

4. The ultrasound system of claim 1, wherein the flow artifact is at least one of a pathline and a streamline, and the modulated property is a thickness of the flow artifact.

5. The ultrasound system of claim 1, wherein the flow artifact is at least one of a pathline, a streamline, and a ribbon, and the modulated property is a length of the flow artifact.

6. The ultrasound system of claim 1, wherein the flow artifact is at least one of a pathline, a streamline, and a ribbon, and the modulated property is an alpha fading of a tail end of the flow artifact.

7. The ultrasound system of claim 1, wherein the flow artifact is at least one of a pathline, a streamline, and a ribbon, and the flow artifact is based at least in part on a time to live duration.

8. The ultrasound system of claim 7, wherein the time to live duration is based at least in part on a velocity of the liquid.

9. The ultrasound system of claim 1, wherein the flow artifact is based at least in part on a selection window.

10. The ultrasound system of claim 1, wherein the object is a patient and the liquid is the blood of the patient.

11. A method for visualizing the flow of a liquid within an object comprising:
obtaining ultrasound data from the object via an ultrasound probe;
rendering one or more images based at least in part on the ultrasound data via a generating unit operatively connected to the ultrasound probe, each image including a flow artifact that depicts the flow of the liquid;
displaying the one or more rendered images on a display processing unit operatively connected with the generating unit; and
modulating a property of the flow artifact based at least in part on a flow characteristic of the liquid.

12. The method of claim 11, wherein the flow artifact is at least one of a pathline, a streamline, a ribbon, and a contour map.

13. The method of claim 11, wherein the flow characteristic of the liquid is at least one of velocity direction, velocity magnitude, acceleration direction, acceleration magnitude, power, turbulence, and strain.

14. The method of claim 11, wherein the flow artifact is at least one of a pathline and a streamline, and the modulated property is a thickness of the flow artifact.

15. The method of claim 11, wherein the flow artifact is at least one of a pathline, a streamline, and a ribbon, and the modulated property is a length of the flow artifact.

16. The method of claim 11, wherein the flow artifact is at least one of a pathline, a streamline, and a ribbon, and the modulated property is an alpha fading of a tail end of the flow artifact.

17. The method of claim 11 further comprising:
determining a time to live duration via the generating unit, and
wherein the flow artifact is at least one of a pathline, a streamline, and a ribbon, and the flow artifact is based at least in part on the time to live duration.

18. The method of claim 17, wherein determining a time to live duration via the generating unit is based at least in part a velocity of the liquid.

19. The method of claim 11 further comprising:
selecting a selection window; and
wherein the flow artifact is based at least in part on the selection window.

20. A non-transitory computer readable medium storing instructions configured to adapt a generating unit, of an ultrasound system for visualizing a liquid within an object, that includes at least one processor and a memory device to:
obtain ultrasound data from the object via an ultrasound probe operatively connected with the generating unit;
render one or more images based at least in part on the ultrasound data, each image including a flow artifact that depicts the flow of the liquid; and
modulate a property of the flow artifact based at least in part on a flow characteristic of the liquid;
display the one or more rendered images on a display processing unit operatively connected with the generating unit; and
wherein the flow artifact is at least one of a pathline, a streamline, a ribbon, and a contour map, and
the flow characteristic of the liquid is at least one of velocity direction, velocity magnitude, acceleration direction, acceleration magnitude, power, turbulence, and strain.
